(19) United States
(12) Patent Application Publication
(10) Pub. No.: US 2013/0097768 A1
(43) Pub. Date: Apr. 25, 2013

(54) Ventilated goggles

(71) Applicant: Anthony Wang Lee, Tainan City (TW)

(72) Inventor: Anthony Wang Lee, Tainan City (TW)

(21) Appl. No.: 13/277,344

(22) Filed: Oct. 20, 2011

(51) Int. Cl. A61F 9/02

(52) U.S. Cl. 2/436

(57) ABSTRACT

A pair of ventilated goggles has a frame body, a lens and a strap. The frame body includes a rigid frame and a resilient frame. The rigid frame has at least one air passage, at least one retaining wall and a cover board. The at least one air passage is formed through the rigid frame. The at least one retaining wall extends from the rigid frame along one of the at least one air passage. The cover board extends from the rigid frame and is positioned over the at least one air passage and the at least one retaining wall. The resilient frame is formed to the rigid frame. The lens is mounted in the frame body. The headband includes two ends respectively connected to two sides of the frame body. With the above-mentioned structure, an enhanced ventilation effect and greater structural strength can be achieved.

### BACKGROUND OF THE INVENTION

1. Field of the Invention

The present invention relates to a pair of goggles, and more particularly to a pair of ventilated goggles that provides an enhanced ventilation effect and increased structural strength.

2. Description of the Prior Arts

A pair of goggles is a form of eye protection that is used to enclose the area surrounding a user's eyes to prevent particulates, chemicals or other hazardous substances from striking the eyes during industrial operations or chemical experiments.

A conventional pair of goggles comprises a frame, a lens and a strap. The lens is mounted in the frame. The strap includes two ends respectively connected to two sides of the frame. For a user to wear the goggles, the strap is fitted around the head of the user and the frame is urged against the face of the user so as to make the lens positioned in front of the eyes of the user.

However, the frame of the conventional pair of goggles is made of resilient material that is not strong enough and is easily deformed and damaged. Besides, heat and sweat easily build up inside the frame and fog the lens due to a lack of suitable ventilation, and thereby causes a moist, muggy environment inside the frame that may lead to eyes discomfort.

To overcome the shortcomings, the present invention provides a pair of ventilated goggles to mitigate or obviate the aforementioned problems.

### SUMMARY OF THE INVENTION

The main object of the present invention is to provide a pair of ventilated goggles that provides an enhanced ventilation effect and increased structural strength.

To achieve the foregoing objective, the pair of ventilated goggles in accordance with the present invention comprises a frame body, a lens and a strap. The frame body includes a rigid frame, a resilient frame and a receiving space. The rigid frame has a rear, a top portion, a mounting opening, at least one air passage, at least one retaining wall and a cover board. Each one of the at least one air passage is formed through the top portion of the rigid frame. Each one of the at least one retaining wall extends upward from the top portion of the rigid frame along a back edge of one of the at least one air passage. The cover board extends upward from a front edge of the top portion of the rigid frame and is positioned over the at least one air passage and the at least one retaining wall. The resilient frame is formed to the rear of the rigid frame and has a back panel extending upward from a top rear edge thereof The receiving space is defined between the at least one retaining wall of the rigid frame and the back panel of the resilient frame and communicates with the at least one air passage. The lens is mounted in the mounting opening of the frame body. The headband includes two ends respectively connected to two sides of the frame body. With the above-mentioned structure, an enhanced ventilation effect and greater structural strength can be achieved.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a pair of ventilated goggles in accordance with the present invention;

FIG. 2 is a partially enlarged cross-sectional perspective view of the pair of ventilated goggles in FIG. 1; and

FIG. 3 is an enlarged bottom perspective view of a rigid frame of the pair of ventilated goggles in FIG. 1.

### DETAILED DESCRIPTION OF THE PREFERRED EMBODIMENTS

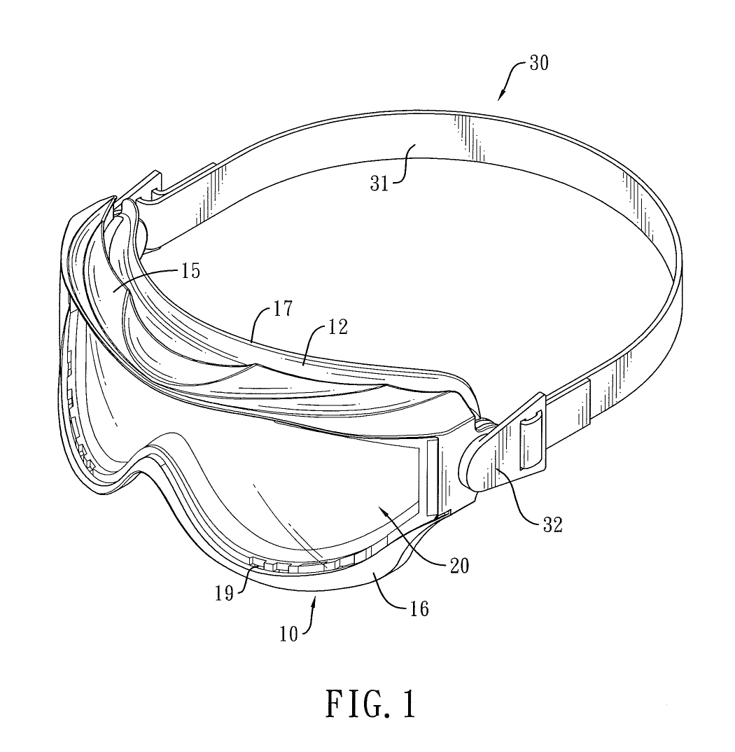

With reference to FIG. 1, a pair of ventilated goggles in accordance with the present invention comprises a frame body 10, a lens 20 and a headband 30.

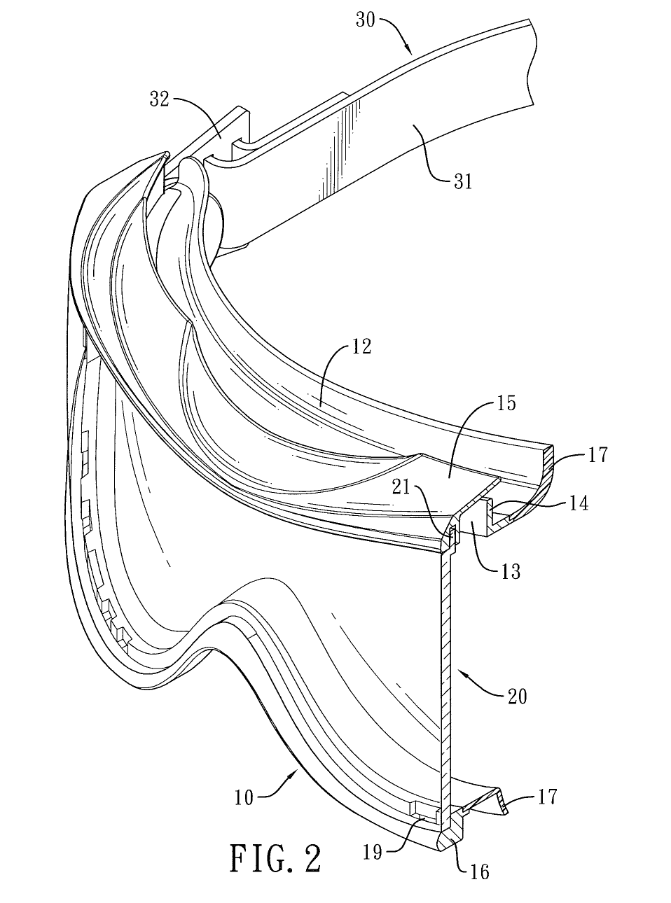

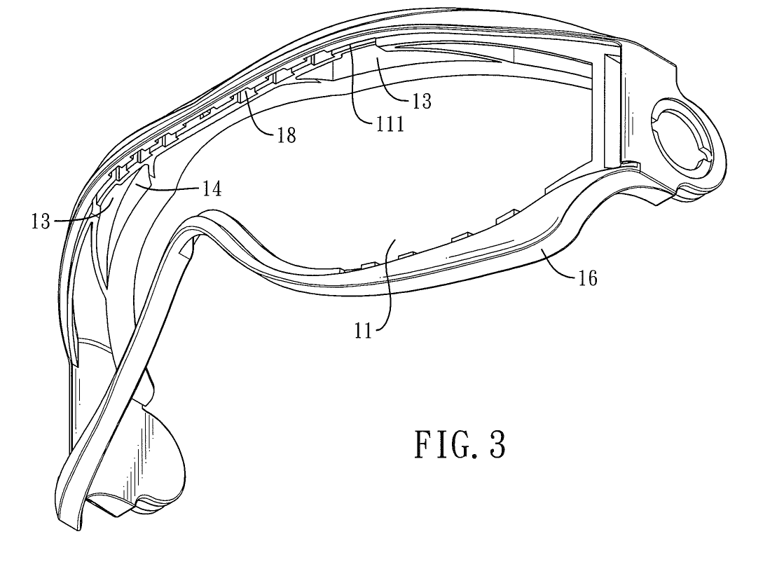

The frame body 10 includes two opposite sides, a rigid frame 16, a resilient frame 17 and a receiving space 12. With further reference to FIGS. 2 and 3, the rigid frame 16 has a rear, an inner surface, a top portion, a bottom portion, a mounting opening 11, a mounting groove 111, at least one air passage 13, at least one retaining wall 14, a cover board 15 and multiple auxiliary air passages 19. The mounting opening 11 is defined in the frame body 10. The mounting groove 111 is formed in a front portion of the inner surface of the rigid frame 16, communicates with the mounting opening 11 and has two clamping surfaces 18. The clamping surfaces 18 are uneven and are formed on opposite groove surfaces of the mounting groove 111. Each one of the at least one air passage 13 is formed through the top portion of the rigid frame 16 and may be angular. Each one of the at least one retaining wall 14 extends upward from the top portion of the rigid frame 16 along a back edge of one of the at least one air passage 13. The cover board 15 extends upward from a front edge of the top portion of the rigid frame 16 and is positioned over the at least one air passage 13 and the at least one retaining wall 14. The auxiliary air passages 19 are respectively formed through the bottom portion of the rigid frame 16 and communicate with the mounting opening 11.

With reference to FIGS. 1 and 2, the resilient frame 17 is formed to the rear of the rigid frame 16 by co-injection molding and has a back panel extending upward from a top rear edge thereof. The receiving space 12 is defined between the at least one retaining wall 14 of the rigid frame 16 and the back panel of the resilient frame 17 and communicates with the at least one air passage 13.

The lens 20 is mounted in the mounting opening 11 of the frame body 10 and includes a peripheral edge and multiple inserting portions 21. The inserting portions 21 respectively extend from the peripheral edge of the lens 20, are inserted into the mounting groove 111 of the rigid frame 16 of the frame body 10 and are securely clamped by the clamping surfaces 18 of the mounting groove 111.

The headband 30 is connected to the frame body 10 and includes a strap 31 and two connecting units 32. The strap 31 has two ends respectively connected to the connecting units 32. The connecting units 32 are respectively mounted on the sides of the frame body 10.

For a user to wear the goggles, the strap 31 of the headband 30 is fitted around the head of the user and the resilient frame 17 of the frame body 10 is urged against the face of the user so as to make the lens 20 positioned in front of the eyes of the user.

When the user wearing the goggles is performing industrial operations or chemical experiments, particulates, chemicals or other hazardous substances will strike the lens 20 and the cover board 15 of the frame body 10 or further fall into the receiving space 12 of the frame body 10. The hazardous substances will not enter the air passage 13 of the frame body 10 because of blocking by the cover board 15 and the retaining wall 14 of the frame body 10. As a result, the user's eyes can be well protected.

Besides, air can flow directly into the frame body 10 through the air passage 13 and the auxiliary air passages 19 of the frame body 10, and thereby heat and sweat will not build up inside the frame body 10 and fog the lens 20 and the user's eyes can be protected from a moist, muggy environment.

Moreover, the frame body 10 is composed of the rigid frame 16 and the resilient frame 17, the rigid frame 16 can structurally reinforce the frame body 10 and prevent deformation and damage, and the resilient frame 17 can provide a comfortable feel to the user's face.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A pair of ventilated goggles comprising:
a frame body including
two opposite sides;
a rigid frame having
a rear;
a top portion;
a mounting opening defined in the frame body;
at least one air passage formed through the top portion of the rigid frame;
at least one retaining wall extending upward from the top portion of the rigid frame along a back edge of one of the at least one air passage; and
a cover board extending upward from a front edge of the top portion of the rigid frame and positioned over the at least one air passage and the at least one retaining wall;
a resilient frame formed to the rear of the rigid frame and having a back panel extending upward from a top rear edge thereof; and
a receiving space defined between the at least one retaining wall of the rigid frame and the back panel of the resilient frame and communicating with the at least one air passage;
a lens mounted in the mounting opening of the frame body; and
a headband including two ends respectively connected to the sides of the frame body.

2. The pair of ventilated goggles as claimed in claim 1, wherein the at least one air passage of the frame body is angular.

3. The pair of ventilated goggles as claimed in claim 2, wherein
the rigid frame of the frame body includes
an inner surface; and
a mounting groove formed in a front portion of the inner surface of the rigid frame, communicating with the mounting opening and having two clamping surfaces being uneven; and
the lens includes multiple inserting portions respectively extending from a peripheral edge thereof, inserted into the mounting groove of the rigid frame of the frame body and securely clamped by the clamping surfaces of the mounting groove.

4. The pair of ventilated goggles as claimed in claim 1, wherein the headband includes a strap having two ends respectively connected to two connecting units, and the connecting units are respectively mounted on the sides of the frame body.

5. The pair of ventilated goggles as claimed in claim 2, wherein the headband includes a strap having two ends respectively connected to two connecting units, and the connecting units are respectively mounted on the sides of the frame body.

6. The pair of ventilated goggles as claimed in claim 3, wherein the headband includes a strap having two ends respectively connected to two connecting units, and the connecting units are respectively mounted on the sides of the frame body.
